(19) United States
(12) Patent Application Publication
(10) Pub. No.: US 2022/0013232 A1
(43) Pub. Date: Jan. 13, 2022

(54) ARTIFICIAL INTELLIGENCE ASSISTED PHYSICIAN SKILL ACCREDITATION

(71) Applicant: Welch Allyn, Inc., Skaneateles Falls, NY (US)

(72) Inventors: Johannes de Bie, Monte San Pietro (IT); Patrick James Noffke, Hartland, WI (US)

(21) Appl. No.: 17/304,780

(22) Filed: Jun. 25, 2021

Related U.S. Application Data
(60) Provisional application No. 63/049,308, filed on Jul. 8, 2020.

(51) Int. Cl. G16H 50/20; G16H 50/30; G16H 10/60

(52) U.S. Cl. CPC G16H 50/20; G16H 10/60; G16H 50/30

(57) ABSTRACT

A diagnostic exam review system is configured to assign queues of diagnostic exams for interpretation by medical professionals. The diagnostic exams are reviewed and recorded in patient files. Some of the exams are reviewed by a second medical professional to determine if the interpretations match. The medical professionals are scored based on whether their interpretations agree, indicating their skill in interpreting a particular type of diagnostic exam. The scores are used to rank medical professionals based on their level of skill.

### BACKGROUND

Complex diagnostic exams, such as X-rays or electrocardiograms, often require specialist physician review. The physician reviews the image or signal waveforms, performs measurements and provides an interpretation and conclusion of the examination. Often, many examinations are reviewed consecutively, guided by an automated system that produces worklists and assigns them to physicians on a rotating schedule. Currently, many of these systems also offer an initial computer interpretation, measurements and conclusions by an automatic algorithm, which aids the physicians in their review.

### SUMMARY

In one aspect, an example diagnostic exam review system can include: a diagnostic exam data store; a medical professional performance data store; at least one processor; and at least one system memory encoding instructions which, when executed by the at least one processor, cause the system to: assign queues of diagnostic exams for each of a plurality of medical professionals to interpret, the diagnostic exams including both unreviewed diagnostic exams and reviewed diagnostic exams; communicate the queues of diagnostic exams to computing devices operated by each of the plurality of medical professionals; receive interpretations for each of the diagnostic exams from the computing devices operated by the medical professionals; analyze the interpretations of the diagnostic exams to determine whether at least two medical professionals' interpretations are consistent with one another; and score the diagnostic exams based on the analysis.

In another aspect, an example computer-implemented method of managing medical professional skill accreditation can include: receiving a diagnostic exam at a diagnostic exam review system from a data source; assigning the diagnostic exam to a first medical professional for interpretation; communicating the diagnostic exam to a computing device operated by the first medical professional; receiving a primary interpretation of the diagnostic exam at the diagnostic exam review system from the computing device operated by the first medical professional; recording the primary interpretation of the diagnostic exam in an electronic medical record system; determining whether the diagnostic exam should be over-read for scoring on the primary interpretation; when the diagnostic exam should be over-read, assigning the diagnostic exam to a second medical professional for interpretation; communicating the diagnostic exam to a computing device operated by the second medical professional; receiving a primary interpretation of the diagnostic exam at the diagnostic exam review system from the computing device operated by the second medical professional; determining, at the diagnostic exam review system, if the primary interpretation of the first medical professional agrees with the primary interpretation of the second medical professional; when the primary interpretations of the first medical professional and second medical professional disagree, the diagnostic exam is assigned to a third medical professional for interpretation; communicating the diagnostic exam to a computing device operated by the third medical professional; receiving a primary interpretation of the diagnostic exam at the diagnostic exam review system from the computing device operated by the third medical professional; determining, at the diagnostic exam review system, if the primary interpretation of the first medical professional agrees with the primary interpretation of the third medical professional; scoring each medical professional's primary interpretation; and recording the scores in a medical professional performance data store.

In yet another aspect, an example non-transitory machine-readable storage medium, comprising computer executable instructions that, when executed by a computing system, can cause the computing system to perform a method including: receiving a diagnostic exam at a diagnostic exam review system from a data source; assigning the diagnostic exam to a first medical professional for interpretation; communicating the diagnostic exam to a computing device operated by the first medical professional; receiving a primary interpretation of the diagnostic exam at the diagnostic exam review system from the computing device operated by the first medical professional; recording the primary interpretation of the diagnostic exam in an electronic medical record system; determining whether the diagnostic exam should be over-read for training or scoring on the primary interpretation; when the diagnostic exam should be over-read, assigning the diagnostic exam to a second medical professional for interpretation; communicating the diagnostic exam to a computing device operated by the second medical professional; receiving a primary interpretation of the diagnostic exam at the diagnostic exam review system from the computing device operated by the second medical professional; determining, at the diagnostic exam review system, if the primary interpretation of the first medical professional agrees with the primary interpretation of the second medical professional; when the primary interpretations of the first medical professional and second medical professional disagree, the diagnostic exam is assigned to a third medical professional for interpretation; communicating the diagnostic exam to a computing device operated by the third medical professional; receiving a primary interpretation of the diagnostic exam at the diagnostic exam review system from the computing device operated by the third medical professional; determining, at the diagnostic exam review system, if the primary interpretation of the first medical professional agrees with the primary interpretation of the third medical professional; scoring each medical professional's primary interpretation; recording the scores in a medical professional performance data store; ranking each of the medical professionals based on the scores and recording the ranking in the medical professional performance data store.

In yet another aspect, an example method of training a machine learning system for reviewing diagnostic exams can include: determining scores and rankings of a plurality of medical professionals for reviewing the diagnostic exams; selecting reviewed diagnostic exams reviewed by a subgroup of the plurality of medical professionals having the highest rankings; and training a machine learning algorithm with the reviewed diagnostic exams.

In yet another aspect, an example method of validating a machine learning system for reviewing diagnostic exams can include: determining scores and rankings of a plurality of medical professionals for reviewing the diagnostic exams; selecting diagnostic exams reviewed by two or more of the subgroup of the plurality of medical professionals having the highest rankings; and validating the machine learning algorithm with the diagnostic exams reviewed by two or more of the subgroup of the plurality of medical professionals having the highest rankings.

### DESCRIPTION OF THE FIGURES

The following drawing figures, which form a part of this application, are illustrative of described technology and are not meant to limit the scope of the disclosure in any manner.

FIG. 1 is a schematic block diagram illustrating an example medical data system.

FIG. 2 is a detailed schematic diagram of the diagnostic exam review system of FIG. 1.

FIG. 3 is a flow diagram of an example method of managing physician skill accreditation.

FIG. 4 is a flow chart illustrating one method of scoring diagnostic exams.

FIG. 5 is a flow chart illustrating another method of scoring diagnostic exams.

FIG. 6 is a block diagram illustrating example components of a computing device usable in the system of FIG. 1.

### DETAILED DESCRIPTION

The term “medical professional” as used herein refers to any licensed professional that is engaged in analyzing examination data, such as waveforms or images, in order to diagnose and treat patients. Medical professionals can include, for example, physicians, pathologists, and nurse practitioners. The term “reader” is also used to describe medical professionals that are reviewing diagnostic examinations.

The term “diagnostic examination” as used herein refers to any examination, image, or sample that can be reviewed by a medical professional on a computing device. Examples include electrocardiograms, echocardiograms, x-rays, and pathology slides.

The term “interpretation” as used herein refers to a result, diagnosis, or conclusion that a medical professional develops as the result of reviewing or reading a diagnostic examination. In some embodiments, the interpretation can be made by a computer program using machine learning techniques. Generally, the term “interpretation” refers to making a yes/no determination about one aspect of an exam at a time. The term “over-read” is also used to refer to an interpretation.

The embodiments described herein aim to provide healthcare institutions with an integrated and automatic metric of the quality of their medical professionals' over-reading skills, providing continuous “accreditation” as well as identifying areas in which medical professionals may require more training. In addition, the system, over time, creates a reliable database of exams. These exams have been read by multiple experts and are suitable to be used to train and validate machine learning algorithms for exam interpretation. Eventually clinical evidence will be generated that will indicate whether and where artificial intelligence (AI) or machine learning (ML) systems may actually replace the medical professional's over-read.

Embodiments disclosed herein can continuously select specific exams out of the normal workflow for over-read by multiple medical professionals. Generally, only the first one of the results is used clinically for diagnosing or treating the patient. The additional readings are exclusively used for quality metrics. In some embodiments, first reads of exams can also be used for quality metrics. The exams selected for over-read are interspersed within the routine workflow and appear identical to normal exams to the over-reading medical professional.

In some embodiments, an algorithm selects to over-read more difficult cases, or cases in which the automatic initial interpretation is ambiguous or close to set thresholds. The algorithm may also randomly select and assign cases (regardless of automatic interpretation algorithm certainty or scoring metrics) in order to encompass undiscovered biases in the automatic interpretation algorithm. It then assigns these to medical professionals randomly, or, as the case may be, to medical professionals who have reviewed few exams with similar characteristics. In some embodiments, the number of additional reviews included in a typical workflow is about 10% of the normal workload. In some embodiments, the number of over-reads may be reduced in an established and mature system.

In one embodiment of the basic principle, each exam selected as a metric is presented to two readers (A and B) instead of the normal single over-read. If both readers agree with each other on the interpretation, the over-reads are complete and scores do not change. If the readers do not agree, the same exam will be presented to a third reader, C. The scores are now adjusted based on whether reader C agrees with A or B.

In an alternate embodiment of the basic principle, each exam selected as a metric is presented to three readers instead of the normal single over-read. If all readers agree with each other on the interpretation, the scores do not change. If only two readers agree, the scores are adjusted based on which pair agrees. In some embodiments, this concept can be expanded to five readers, seven readers, or any odd number of readers.

The algorithm selects the medical professionals from the hospital pool on the basis of how many tests they have already executed in a category, such that all reviewer triplet combinations (e.g. A, B and C) occur in similar numbers. When enough tests have been executed, the scores of all medical professionals are scaled and compared to provide a ranking.

The integration of the system within a diagnostic exam workflow management system will provide statistical reports to institution management on the quality of the readings and readers. Additionally, the reports may identify gaps for aspects or individuals where specific training may be needed. It may also provide feedback to medical professionals, thus stimulating continuous education. The system is intended to be totally automatic.

The system also creates a database of exam results that can be used for training and validation of automatic algorithms since every test exam has been reviewed by two or more experts. Because the ranking of each reviewer is known for each aspect of the interpretation and the various classes of test outcomes, training of an algorithm may be done on exams reviewed by the highest-ranking medical professionals, thus enabling a best-in-class outcome of the training. In addition, validation of the algorithm may then be performed on those test cases that have been reviewed by multiple medical professionals. The algorithm validation is therefore acceptable from a scientific point of view, and compliant with accepted regulations. In fact, the system thus continuously maintains its own prospective study without additional cost.

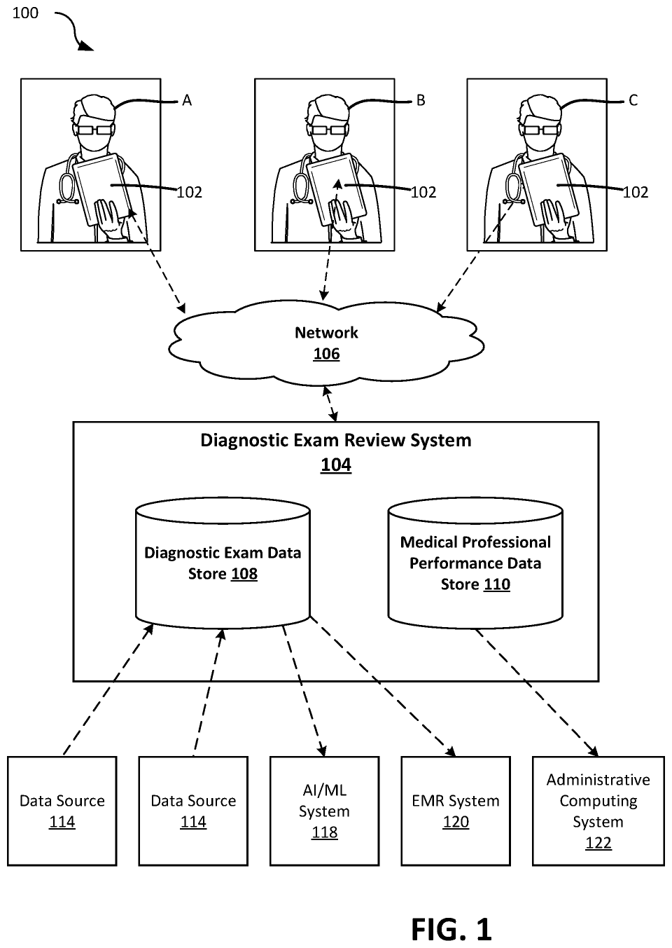

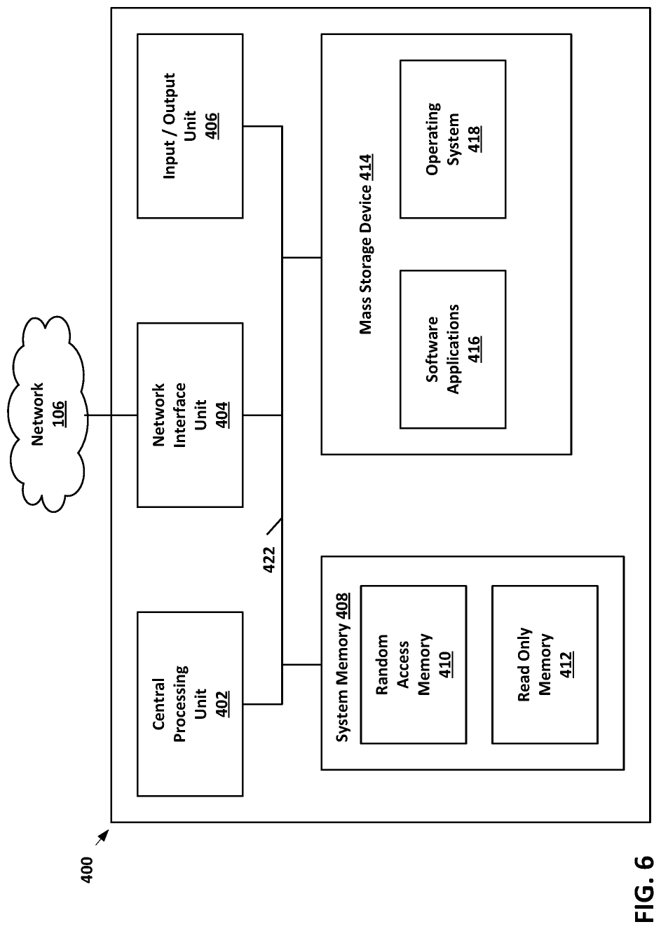

FIG. 1 is a schematic block diagram illustrating an example medical data system 100. The medical data system 100 includes a plurality of computing devices 102 operated by medical professionals (A, B, C). The computing devices 102 can be any device having a processor, memory, communication capability, and a display. In some embodiments, the computing devices 102 are portable devices such as tablet computers, smartphones, or laptop computers. In some embodiments, the computing devices 102 are desktop computers or other stationary computing terminals. Further details of the computing devices 102 are provided in FIG. 6.

The medical professional computing devices 102 communicate with a diagnostic exam review system 104 through a communication network 106. The network 106 may include any type of wireless network, a wired network, or any communication network known in the art. For example, wireless connections can include cellular network connections and connections made using protocols such as 802.11a, b, and/or g.

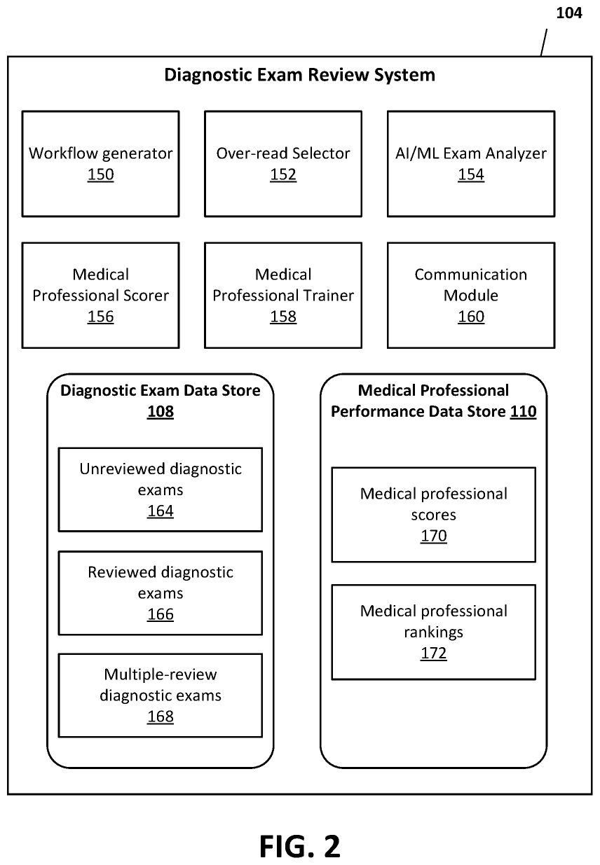

The diagnostic exam review system 104 operates to manage the over-reading of diagnostic exams. Generally, the diagnostic exam review system 104 could operate on a server or other computing system. In some embodiments, the diagnostic exam review system 104 includes a processing device and a memory. In some embodiments, the memory includes data stores and instructions that are executed by the processing device. In some embodiments, the data stores include a diagnostic exam data store 108 and a medical professional performance data store 110. In some embodiments, one or more data stores could be housed external to the diagnostic exam review system 104 and communicate through wired or wireless connections. In some embodiments, the instructions stored in the diagnostic exam review system 104 execute various modules or applications for performing particular tasks. Some of those tasks can include assigning queues of diagnostic exams to medical professionals for review and interpretation, selecting diagnostic exams to be over-read by additional medical professionals, scoring over-read diagnostic exams, and ranking medical professionals based on their over-reading skills. More details about the diagnostic exam review system 104 are provided in FIG. 2.

The diagnostic exam review system 104 receives new diagnostic exams from one or more data sources 114 that need to be interpreted. These unreviewed diagnostic exams can be stored in the diagnostic exam data store 108 until they are interpreted by a medical professional. The diagnostic exam review system 104 assigns the diagnostic exams to medical professionals A, B, C for review. The diagnostic exams are communicated to the computing devices 102 operated by the medical professionals through the network 106. The medical professionals provide their interpretations and communicate the interpretations back to the diagnostic exam review system 104 through the network 106. All of the reviewed diagnostic exams are communicated to an electronic medical record (EMR) system 120 to be recorded in patient records.

Some of the reviewed diagnostic exams are reviewed by at least one additional medical professional. This is done for training purposes and to determine medical professionals' level of skill. Interpretations by two or more medical professionals are compared to determine if they agree on the results of a diagnostic exam. The diagnostic exam review system 104 scores the medical professionals based on their interpretations of the diagnostic exams and stores the scores in the medical professional performance data store 110. Medical professionals can also be ranked based on their scores. Scores can be determined for each of a plurality of aspects of a diagnostic exam as well as for different types of diagnostic exams. The medical professional scores and rankings can be communicated to an administrative computing system 122 for review and reporting.

The diagnostic exam review system 104 can utilize medical professional rankings to aid in selecting reviewed diagnostic exams from the diagnostic exam data store 108 to communicate to an artificial intelligence/machine learning (AI/ML) system 118. The AI/ML system 118 can use the diagnostic exam data to train and validate machine learning algorithms for automatically analyzing diagnostic exams. In some embodiments, the AI/ML system 118 is trained using only multiple-review diagnostic exams in which the reviewers agreed on the interpretation. In some embodiments, the AI/ML system 118 is trained using diagnostic exams that have only been reviewed once, but by medical professionals with high ranks. Either way, the data used to train the AI/ML system 118 is selected to be higher quality in order to produce accurate results.

AI/ML systems 118 could serve to perform initial automatic interpretations of diagnostic exams before a medical professional double-checks the exams. In some embodiments, the AI/ML systems 118 could analyze diagnostic exams and only notify medical professionals of difficult cases that need to be reviewed. In some embodiments, the AI/ML system 118 could eventually perform all interpretations of diagnostic exams.

FIG. 2 is a detailed schematic diagram of the diagnostic exam review system 104 of FIG. 1. In addition to the diagnostic exam data store 108 and medical professional performance data store 110 mentioned in FIG. 1, the diagnostic exam review system 104 includes a workflow generator 150, an over-read selector 152, an AI/ML exam analyzer 154, a medical professional scorer 156, a medical professional trainer 158, and a communication module 160.

The workflow generator 150 operates to prepare and assign queues of diagnostic exams for each of a plurality of medical professionals to interpret. As diagnostic exams are received from data sources, they are assigned to medical professionals to review in batches. The majority of each medical professional's queue is unreviewed diagnostic exams. In some embodiments, a number of previously reviewed diagnostic exams are included in the queue. In some embodiments, the over-read selector 152 selects which of the reviewed exams in the diagnostic exam data store 108 are selected to be included in each medical professional's queue. In some embodiments, each queue includes at least 1% previously reviewed diagnostic exams in addition to the unreviewed diagnostic exams. In some embodiments, each queue includes at least 2%, at least 3%, at least 4%, or at least 5% previously reviewed diagnostic exams. In some embodiments, each queue includes from about 5% to about 15% previously reviewed diagnostic exams.

The over-read selector 152, as mentioned above, operates to select previously reviewed diagnostic exams to be over-read by medical professionals. In some embodiments, the diagnostic exams are selected at random. In some embodiments, the diagnostic exams are selected because they are difficult to interpret, thereby providing quality training opportunities for the medical professionals. In some embodiments, the over-read selector 152 selects particular types of diagnostic exams for a particular medical professional to over-read in order to improve the medical professional's skills in interpreting that type of diagnostic exam. In some embodiments, exams are selected in order to balance the number of exams the reader has seen in order to score and rank the reader on different aspects of exam interpretation.

The AI/ML exam analyzer 154 operates to analyze diagnostic exams using algorithms trained on sample diagnostic exam data. In some embodiments, the AI exam analyzer 154 draws upon the diagnostic exam data store 108 to continually update its algorithms, thus continually improving its quality. In some embodiments, the AI exam analyzer 154 analyzes each unreviewed diagnostic exam before it is put into a medical professional's queue. An initial automatic interpretation is provided with the unreviewed diagnostic exam when it is presented to the medical professional. In some embodiments, the AI exam analyzer 154 automatically determines an interpretation for some diagnostic exams and those exams are not reviewed by a medical professional because the outcome is clear based on the AI exam analyzer 154 analysis. Other diagnostic exams are flagged by the AI exam analyzer 154 as requiring review by a medical professional and then they are included in the queues. In some embodiments, the AI exam analyzer 154 operates in conjunction with the over-read selector 152 to select diagnostic exams for over-read based on how ambiguous the AI exam analyzer 154 determines the diagnostic exam to be.

The medical professional scorer 156 operates to analyze over-read diagnostic exams received from medical professional computing devices 102. The interpretations of each medical professional for a single diagnostic exam are compared for one or more aspects of the exam. In some embodiments, the diagnostic exam requires both a primary interpretation and a secondary interpretation. One example of such a diagnostic exam is an interpretation of an ECG as “sinus rhythm with first-degree AV block,” which is described further below.

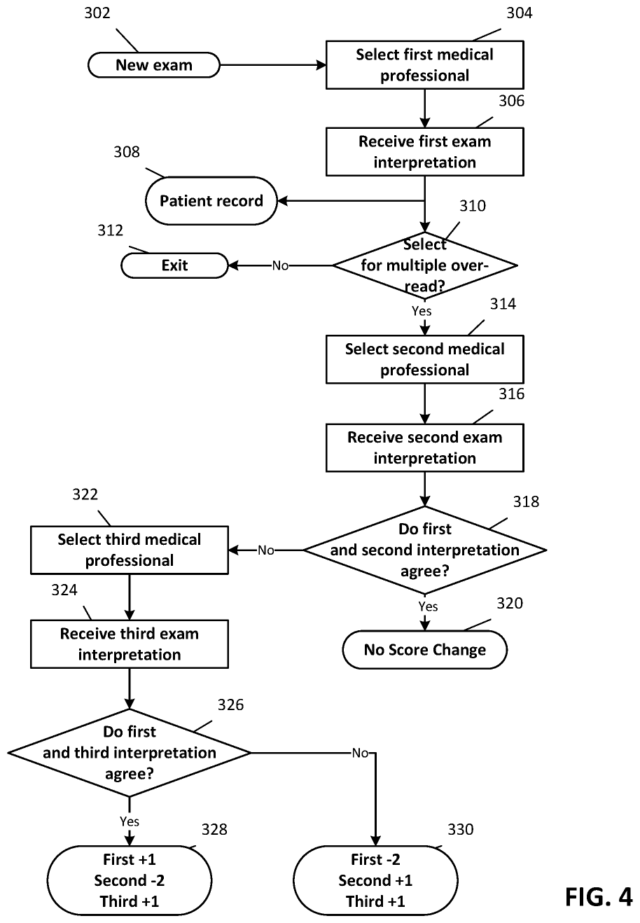

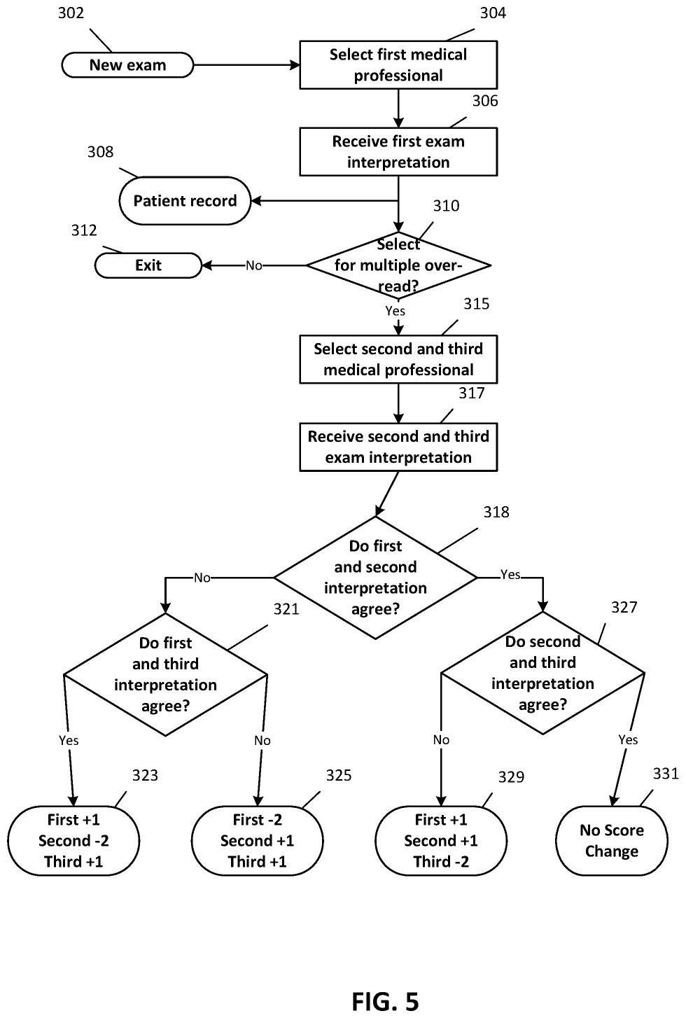

The medical professional scorer 156 awards points to each medical professional when their interpretations match other medical professionals' interpretations. Examples of scoring exams are provided in FIGS. 4 and 5. Overall scores for each medical professional are used to generate rankings of the medical professionals. In some embodiments, the rankings are categorized by type of diagnostic exam. In some embodiments, the rankings can be broken down for each aspect of a particular diagnostic exam. In some embodiments, the medical professional scorer 156 also functions to generate reports of medical professional scores and rankings.

The medical professional trainer 158 operates to identify medical professionals that require additional training in one or more aspects of one or more types of diagnostic exam. In some embodiments, scores and rankings provided by the medical professional scorer 156 are used to inform decisions about which medical professionals need additional training. In some embodiments, the medical professional trainer 158 communicates with the over-read selector 152 and workflow generator 150 to select appropriate numbers and types of diagnostic exams to be assigned to particular medical professionals for over-reading.

The communication module 160 operates to manage communication of data in and out of the diagnostic exam review system as well as within the diagnostic exam review system.

The diagnostic exam data store 108 operates to store diagnostic exams utilized by the diagnostic exam review system. In some embodiments, the diagnostic exam data store 108 includes unreviewed diagnostic exams 164, reviewed diagnostic exams 166, and multiple-review diagnostic exams 168.

In some embodiments, the unreviewed diagnostic exams 164 are exams that have not yet been interpreted by a medical professional. In some embodiments, unreviewed diagnostic exams 164 include exams that have been analyzed with AI, but not by a medical professional. In some embodiments, unreviewed diagnostic exams 164 exclude those exams that have been analyzed by AI.

In some embodiments, the reviewed diagnostic exams 166 are exams that have been interpreted by a medical professional. In some embodiments, reviewed diagnostic exams 166 have also been reviewed by an AI system. In some embodiments, reviewed diagnostic exams 166 include exams that have only been reviewed by an AI system.

In some embodiments, the multiple-review diagnostic exams 168 are exams that have been interpreted by at least two different medical professionals. In some embodiments, multiple-review diagnostic exams 168 can include exams that were initially analyzed by an AI system and were over-read by a medical professional.

The medical professional performance data store 110 includes medical professional scores 170 and medical professional rankings 172. In some embodiments, scores and rankings are received from the medical professional scorer 156 and are stored for later use. In some embodiments, the medical professional performance data store 110 can further store reports generated by the medical professional scorer 156.

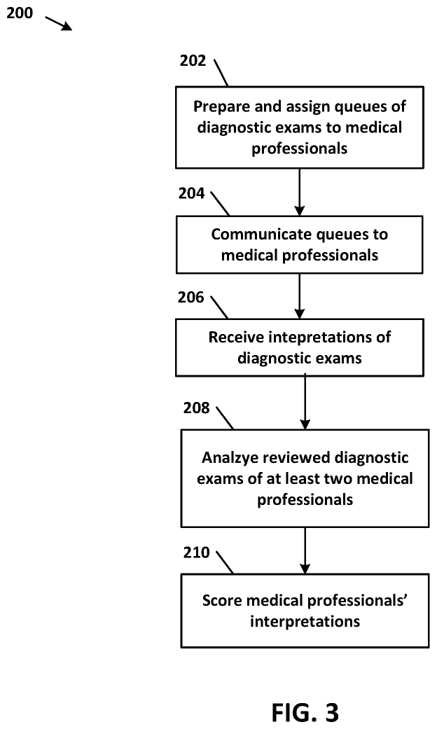

FIG. 3 is a flow diagram of an example method 200 of managing medical professional skill accreditation. In some embodiments, this method 200 is performed by one or more components of the diagnostic exam review system 104 of FIG. 1.

At operation 202, queues of diagnostic exams are prepared and assigned for each of a plurality of medical professionals to interpret. The diagnostic exams include both unreviewed diagnostic exams and reviewed diagnostic exams. In some embodiments, medical professionals are scheduled to review batches of diagnostic exams on a rotating basis. In some embodiments, the diagnostic exams include new, unreviewed exams that have been received during the time period that a medical professional is assigned to review exams. Additionally, exams that were already reviewed are interspersed with the unreviewed exams to provide validation of the results as well as provide training, scoring, and ranking for the medical professional. This process is referred to as “over-reading.”

At operation 204, the queues of diagnostic exams are communicated to computing devices operated by each of the plurality of medical professionals. In some embodiments, the diagnostic exams are sent to the medical professional's computing device via a secure link, encrypted email, or other means communication that are compliant with patient privacy laws (e.g. HIPAA). In some embodiments, the diagnostic exams are reviewed on a graphical user interface of a particular software application or program operating on the medical professional's computing device. The diagnostic exams that have been previously reviewed look like unreviewed diagnostic exams when the medical professional views the exams on a computing device.

At operation 206, interpretations for each of the diagnostic exams are received from the computing devices operated by the medical professionals. The interpretations of the reviewed diagnostic exams are stored in a data store. In some embodiments, reviewed diagnostic exams are communicated to an electronic medical record (EMR) system to be recorded with the appropriate patient files.

At operation 208, the reviewed diagnostic exams are analyzed to determine whether at least two medical professionals' interpretations are consistent with one another. Each diagnostic exam is interpreted by at least two different medical professionals for scoring purposes. In some embodiments, if two medical professionals agree, the diagnostic exam will not be reviewed again. In some embodiments, if two medical professionals disagree, a third medical professional will be assigned to interpret the diagnostic exam.

At operation 210, the medical professionals' interpretations of the diagnostic exams are scored. In some embodiments, zero points are awarded to each medical professional when two or more interpretations of the same diagnostic exam are in agreement. In some embodiments, when three or more medical professionals interpret the same diagnostic exam, positive points are awarded to the majority in agreement and negative points are awarded to the minority in disagreement. Details of two scoring methodologies are described in greater detail in FIGS. 4 and 5 below.

### Scoring

The basic principle of the scoring has been designed for exams, or certain aspects of exams, with only two possible exclusive outcomes (e.g. normal or abnormal). If an exam or certain aspects of the exam have more than two outcomes that are mutually exclusive, the scoring is repeated for all outcomes. For example, in the case of the rhythm of an ECG, there can be a number of rhythm types (e.g. sinus rhythm, atrial fibrillation, electronically paced rhythm) which cannot co-exist. In those cases, the scoring will be repeated for each rhythm type (sinus rhythm vs no sinus rhythm, atrial fibrillation vs no atrial fibrillation and paced rhythm vs no paced rhythm). The scoring has been designed such that, in cases where all readers agree or disagree, the readers' net scores do not change.

All medical professionals start with a default score, such as 1000. The algorithm maintains a “zero-sum” scoring system, such that the sum (and thus the average) of all scores is always the same. The rank of each reader is based on the ratio of the reader's score to the average score.

In some cases, there may be “primary” and “secondary” or “derived” interpretations. An example may be the main rhythm type and an occasional arrhythmia in an ECG. The primary interpretation of an ECG could be either “sinus rhythm” or “atrial fibrillation,” but not both. Each primary interpretation has different possible secondary interpretations. For “sinus rhythm” the secondary interpretations could include “with first-degree AV block” and/or “with occasional supraventricular premature complexes” and/or “with frequent ventricular premature complexes.” In this case, all three secondary interpretations could exist at the same time in a single diagnosis. However, the secondary interpretation of “with first-degree AV block” is not possible with a primary interpretation of “atrial fibrillation.”

Readers A and B may agree on primary interpretations but may disagree on secondary interpretations. In such a case, C would read the exam in both embodiments of the basic design. If C agrees with the primary interpretation, no points will be given for the primary interpretation and scoring will proceed for the secondary interpretation. If, however, C disagrees with the primary interpretation, this would result in adding one point to A and B (for the primary interpretation) and subtracting two points from C. For example, A states “sinus rhythm” and B “sinus rhythm with ventricular extrasystole”; they agree on the primary rhythm, but not on the secondary arrhythmia. If C now does not read the primary rhythm as “sinus rhythm”, this would result in adding one point for A and B and subtracting two points for C in the category “sinus rhythm”.

Additionally, some interpretations may preclude scoring on certain other (secondary) interpretation categories. In such cases, the secondary interpretation category will not be scored. For example, if A states “sinus rhythm with first degree AV block” and B “sinus rhythm”, then C states “atrial fibrillation”, the score for first degree AV block does not change since it cannot exist in the presence of atrial fibrillation. The result for the primary rhythm is scored, however.

If there are multiple primary interpretations that are mutually exclusive (such as primary rhythm statements for an ECG), they can count twice as much as secondary interpretations. For example, if A and C both say sinus rhythm and B says atrial fibrillation, A and C will gain one point each for sinus rhythm and one point each for atrial fibrillation, and B will lose two points for sinus rhythm and two points for atrial fibrillation (because B disagrees for both categories). However, if A, B, and C all agree on sinus rhythm (the primary rhythm statement), but B says first degree AV block and A and C do not, then B loses two points and A and C each gain one point for the “first degree AV block” category, while there are no point changes for the primary statement “sinus rhythm” because all readers agree.

If all three (A, B, and C) disagree on a primary interpretation (when more than two possible interpretations exist), the algorithm stops. The net result is zero change to the score for all three (each reader gains and loses the same amount). Other embodiments may proceed to assign two more readers with a goal to find consensus between three readers. In that case, the three readers that agree would each gain two points and the two remaining would each lose three points. This exam may also be assigned a higher difficulty rating since it resulted in at least three different interpretations. This process may be extended to higher odd numbers of readers.

FIG. 4 is a flow chart illustrating one method of scoring diagnostic exams. In this method, a third opinion is only sought if the first and second interpretations are not in agreement, thereby limiting the number of times three readings of the same exam are needed for ranking/scoring purposes.

At operation 302, a new, unreviewed diagnostic exam is received. In some embodiments, the exam could be temporarily stored until it is assigned to a queue.

At operation 304, the diagnostic exam is assigned to a first medical professional. Generally, the medical professional reviewing the new diagnostic exam is assigned to review a batch of exams on a rotating schedule.

At operation 306, a first interpretation of the diagnostic exam is received from the first medical professional. Generally, the interpretation is a yes/no determination about at least one aspect of the diagnostic exam.

At operation 308, the interpretation is recorded in the patient's EMR.

At operation 310, the diagnostic exam is considered for multiple over-read. In some embodiments, the diagnostic exam may be analyzed by, for example, an AI/ML system to determine if the exam is easy, difficult, or average to interpret and selections are made based on the perceived difficulty of interpreting the exam. In some embodiments, exams are selected at random for multiple over-reading. If the diagnostic exam is not selected for multiple over-read, the method proceeds to operation 312 and the exam exits the process after a single read.

For diagnostic exams selected for multiple over-read, the method proceeds to operation 314. A second medical professional is selected to “over-read” the diagnostic examination by providing a second read. In some embodiments, the second medical professional is selected in the same way that the first medical professional is selected (i.e. they are scheduled to review a batch of exams). In some embodiments, the second medical professional is selected based on their level of skill in interpreting that type of exam. The medical professional could be assigned the exam either because the medical professional is highly skilled and will likely provide an accurate interpretation of the exam, because more interpretations of a type of exam are needed to score and rank the medical professional, or because the medical professional requires more training in that type of exam.

At operation 316, a second interpretation of the diagnostic exam is received from the second medical professional.

At operation 318, the first and second interpretations are analyzed to determine if they agree. If the first and second interpretations of the diagnostic exam are in agreement, the method proceeds to operation 320 and the scores for the first and second medical professional remain the same.

If the first and second interpretation do not agree, the method proceeds to operation 322. A third medical professional is selected or assigned to over-read the diagnostic exam. Similar to selecting the second medical professional, the third medical professional could be selected based on a rotation, based on scoring/ranking needs, or based on their level of skill.

At operation 324, a third interpretation of the diagnostic exam is received from the third medical professional.

At operation 326, the first and third interpretations of the diagnostic exam are analyzed to determine if they agree.

If the first and third medical professionals are in agreement, the method proceeds to operation 328. The first and third medical professionals are awarded one point for being in the majority. The second medical professional is in the minority by not agreeing with the first and third medical professionals. Therefore, the second medical professional is scored negative two.

If the first and third medical professionals disagree, the method proceeds to operation 330. The second and third medical professionals are awarded one point for being in the majority. The first medical professional is in the minority by not agreeing with the second and third medical professionals. Therefore, the first medical professional is scored negative two.

FIG. 5 is a flow chart illustrating another method of scoring diagnostic exams. In this method a third interpretation is recorded for the diagnostic exam regardless of whether the first and second interpretations agree. Operations 302, 304, 306, 308, 310, and 312 are identical to the operations described in FIG. 4. Therefore, their descriptions will not be repeated here.

Proceeding from operation 315, a second medical professional and third medical professional are selected to provide a second read of the exam.

At operation 317, second interpretations of the diagnostic exam is received from the second medical professional and the third medical professional.

Proceeding from operation 318, if the first and second interpretations of the diagnostic exam do not agree, the method proceeds to operation 321. The first and third interpretations are compared to determine if they agree.

If the first and third interpretations are in agreement, the method proceeds to operation 323. The first and third medical professionals are awarded one point for being in the majority. The second medical professional is in the minority by not agreeing with the first and third medical professionals. Therefore, the second medical professional is scored negative two.

If the first and third interpretations are not in agreement, the method proceeds to operation 325. The second and third medical professionals are awarded one point for being in the majority. The first medical professional is in the minority by not agreeing with the second and third medical professionals. Therefore, the first medical professional is scored negative two.

Returning to operation 318, if the first and second interpretations of the diagnostic exam are in agreement, the method proceeds to operation 327. The second and third interpretations are compared to determine if they agree.

If the second and third interpretations do not agree, the method proceed to operation 329. The first and second medical professionals are awarded one point for being in the majority. The third medical professional is scored negative two for being in the minority.

If the second and third interpretation agree, the method proceeds to operation 331. All of the medical professionals' interpretations are in agreement. Therefore, there is no change in their scores.

FIG. 6 is a block diagram illustrating an example of the physical components of a computing device 400. The computing device 400 could be implemented in various aspects of the medical data system 100, such as the computing devices 102 operated by the medical professionals. Components of the computing device 400 can also be incorporated into other devices described herein, such as the diagnostic exam review system 104 or the administrative computing system 122.

In the example shown in FIG. 6, the computing device 400 includes at least one central processing unit (“CPU”) 402, a system memory 408, and a system bus 422 that couples the system memory 408 to the CPU 402. The system memory 408 includes a random access memory (“RAM”) 410 and a read-only memory (“ROM”) 412. A basic input/output system that contains the basic routines that help to transfer information between elements within the computing device 400, such as during startup, is stored in the ROM 412. The computing system 400 further includes a mass storage device 414. The mass storage device 414 is able to store software instructions and data.

The mass storage device 414 is connected to the CPU 402 through a mass storage controller (not shown) connected to the system bus 422. The mass storage device 414 and its associated computer-readable storage media provide non-volatile, non-transitory data storage for the computing device 400. Although the description of computer-readable storage media contained herein refers to a mass storage device, such as a hard disk or solid state disk, it should be appreciated by those skilled in the art that computer-readable data storage media can include any available tangible, physical device or article of manufacture from which the CPU 402 can read data and/or instructions. In certain embodiments, the computer-readable storage media comprises entirely non-transitory media. In some embodiments, the mass storage device 414 is stored on a remote system (e.g. the cloud) accessible to the CPU 402 via the network 106.

Computer-readable storage media includes volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the computing device 400.

According to various embodiments, the computing device 400 can operate in a networked environment using logical connections to remote network devices through a network 106, such as a wireless network, the Internet, or another type of network. The computing device 400 may connect to the network 106 through a network interface unit 404 connected to the system bus 422. It should be appreciated that the network interface unit 404 may also be utilized to connect to other types of networks and remote computing systems. The computing device 400 also includes an input/output controller 406 for receiving and processing input from a number of other devices, including a touch user interface display screen, or another type of input device. Similarly, the input/output controller 406 may provide output to a touch user interface display screen or other type of output device.

As mentioned briefly above, the mass storage device 414 and the RAM 410 of the computing device 400 can store software instructions and data. The software instructions include an operating system 418 suitable for controlling the operation of the computing device 400. The mass storage device 414 and/or the RAM 410 also store software instructions, that when executed by the CPU 402, cause the computing device 400 to provide the functionality discussed in this document.

The block diagrams depicted herein are just examples. There may be many variations to these diagrams described therein without departing from the spirit of the disclosure. For instance, components may be added, deleted or modified.

While embodiments have been described, it will be understood that those skilled in the art, both now and in the future, may make various improvements and enhancements can be made. The description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention.

The claimed inventions should not be construed as being limited to any embodiment, example, or detail provided in this application. Regardless whether shown and described in combination or separately, the various features (both structural and methodological) are intended to be selectively included or omitted to produce an embodiment with a particular set of features.

What is claimed is:

1. A diagnostic exam review system comprising:
a diagnostic exam data store;
a medical professional performance data store;
at least one processor; and
at least one system memory encoding instructions which, when executed by the at least one processor, cause the system to:
assign queues of diagnostic exams for each of a plurality of medical professionals to interpret, the diagnostic exams including both unreviewed diagnostic exams and reviewed diagnostic exams;
communicate the queues of diagnostic exams to computing devices operated by each of the plurality of medical professionals;
receive interpretations for each of the diagnostic exams from the computing devices operated by the medical professionals;
analyze the interpretations of the diagnostic exams to determine whether at least two medical professionals' interpretations are consistent with one another; and
score the diagnostic exams based on the analysis.

2. The diagnostic exam review system of claim 1, wherein the diagnostic exam data store comprises:
unreviewed diagnostic exams that have not yet been interpreted by a medical professional;
reviewed diagnostic exams that have been interpreted by one medical professional; and
multiple-review diagnostic exams that have been interpreted by at least two medical professionals.

3. The diagnostic exam review system of claim 1, wherein the medical professional performance data store comprises scores and rankings for medical professionals.

4. The diagnostic exam review system of claim 1, further comprising instructions that cause the system to:
receive a plurality of unreviewed diagnostic exams from one or more data sources; and
store the unreviewed diagnostic exams in the diagnostic exam data store.

5. The diagnostic exam review system of claim 1, further comprising instructions that cause the system to:
record the interpretations of the diagnostic exams in an electronic medical record system;
record the interpretations of the diagnostic exams that have been reviewed by one medical professional in the diagnostic exam data store as reviewed diagnostic exams; and
record the interpretations of the diagnostic exams that have been reviewed by two or more medical professionals in the diagnostic exam data store as multiple-read diagnostic exams.

6. The diagnostic exam review system of claim 1, further comprising instructions that cause the system to:
analyze, with an automated diagnostic exam analyzing system, the unreviewed diagnostic exams.

7. The diagnostic exam review system of claim 1, wherein scoring comprises:
not changing the scores of each medical professional when the interpretations agree; and
adjusting the scores of each medical professional based on which interpretations are in agreement when not all interpretations agree.

8. The diagnostic exam review system of claim 1, wherein the queues of diagnostic exams for multiple over-read include from about 5% to about 15% previously reviewed diagnostic exams.

9. The diagnostic exam review system of claim 1, further comprising instructions that cause the system to:
rank the plurality of medical professionals based on the scores; and
record the ranking in the medical professional performance data store.

10. The diagnostic exam review system of claim 1, further comprising instructions that cause the system to:
identify one or more of the plurality of medical professionals requiring additional training based on the scores; and
report the one or more of the plurality of medical professionals to an administrator computing system, the report including the scores.

11. The diagnostic exam review system of claim 10, further comprising instructions that cause the system to:
assign additional reviewed diagnostic exams to the one or more of the plurality of medical professionals to provide additional training or scoring.

12. The diagnostic exam review system of claim 9, further comprising instructions that cause the system to:
select reviewed and multiple-review diagnostic exams interpreted by medical professionals based on the rank of the medical professionals; and
communicate the reviewed and multiple-review diagnostic exams to an automated diagnostic exam analyzing system.

13. A computer-implemented method of managing medical professional skill accreditation, the method comprising:
receiving a diagnostic exam at a diagnostic exam review system from a data source;
assigning the diagnostic exam to a first medical professional for interpretation;
communicating the diagnostic exam to a computing device operated by the first medical professional;
receiving a primary interpretation of the diagnostic exam at the diagnostic exam review system from the computing device operated by the first medical professional;
recording the primary interpretation of the diagnostic exam in an electronic medical record system;
determining whether the diagnostic exam should be over-read for scoring on the primary interpretation;
when the diagnostic exam should be over-read, assigning the diagnostic exam to a second medical professional for interpretation;
communicating the diagnostic exam to a computing device operated by the second medical professional;
receiving a primary interpretation of the diagnostic exam at the diagnostic exam review system from the computing device operated by the second medical professional;
determining, at the diagnostic exam review system, if the primary interpretation of the first medical professional agrees with the primary interpretation of the second medical professional;
when the primary interpretations of the first medical professional and second medical professional disagree, the diagnostic exam is assigned to a third medical professional for interpretation;
communicating the diagnostic exam to a computing device operated by the third medical professional;
receiving a primary interpretation of the diagnostic exam at the diagnostic exam review system from the computing device operated by the third medical professional;
determining, at the diagnostic exam review system, if the primary interpretation of the first medical professional agrees with the primary interpretation of the third medical professional;
scoring each medical professional's primary interpretation; and
recording the scores in a medical professional performance data store.

14. The method of claim 13, wherein scoring comprises:
awarding the first medical professional and second medical professional zero points when their primary interpretations agree;
awarding the first medical professional one point, the second medical professional negative two points, and the third medical professional one point when the first and third medical professional agree and the second medical professional disagrees with the primary interpretation of the diagnostic exam; and
awarding the first medical professional negative two points, the second medical professional one point, and the third medical professional one point when the second and third medical professional agree and the first medical professional disagrees with the primary interpretation of the diagnostic exam.

15. The method of claim 13, wherein scoring comprises:
awarding the first medical professional one point, the second medical professional negative two points, and the third medical professional one point when the first and third medical professional agree and the second medical professional disagrees with the primary interpretation of the diagnostic exam;
awarding the first medical professional negative two points, the second medical professional one point, and the third medical professional one point when the second and third medical professional agree and the first medical professional disagrees with the primary interpretation of the diagnostic exam;
awarding the first medical professional one point, the second medical professional one point, and the third medical professional negative two points when the first and second medical professional agree and the third medical professional disagrees with the primary interpretation of the diagnostic exam; and
awarding the first medical professional, second medical professional, and third medical professional zero points when their primary interpretations agree.

16. The method of claim 13, further comprising:
receiving a secondary interpretation of the diagnostic exam at the diagnostic exam review system from the computing device operated by the first medical professional, wherein the secondary interpretation is directed to a different aspect of the diagnostic exam than the primary interpretation;
recording the secondary interpretation of the diagnostic exam in an electronic medical record system;
determining whether diagnostic exam should be over-read for training on the secondary interpretation;
if the diagnostic exam should be over-read for training, assigning the diagnostic exam to a second medical professional for secondary interpretation;
communicating the diagnostic exam to a computing device operated by the second medical professional;
receiving a secondary interpretation of the diagnostic exam at the diagnostic exam review system from the computing device operated by the second medical professional;
determining, at the diagnostic exam review system, if the secondary interpretation of the first medical professional agrees with the secondary interpretation of the second medical professional;
if the secondary interpretations of the first medical professional and second medical professional disagree, the diagnostic exam is assigned to a third medical professional for interpretation;
communicating the diagnostic exam to a computing device operated by the third medical professional;
receiving a secondary interpretation of the diagnostic exam at the diagnostic exam review system from the computing device operated by the third medical professional;
determining, at the diagnostic exam review system, if the secondary interpretation of the first medical professional agrees with the primary interpretation of the third medical professional;
scoring each medical professional's secondary interpretation; and
recording the scores in a medical professional performance data store.

17. The method of claim 13, further comprising ranking each of the medical professionals based on the scores and recording the ranking in the medical professional performance data store.

18. The method of claim 13, further comprising:
identifying one or more of the plurality of medical professionals requiring additional training based on the scores;
reporting the one or more of the plurality of medical professionals to an administrator computing system; and
assigning additional reviewed diagnostic exams to the one or more of the plurality of medical professionals to provide additional training.

19. The method of claim 17, further comprising:
selecting reviewed and multiple-reviewed diagnostic exams interpreted by medical professionals based on the rank of the medical professionals; and
communicating the over-read diagnostic exams to an artificial intelligence diagnostic exam analyzing system.

20. A non-transitory machine-readable storage medium, comprising computer executable instructions that, when executed by a computing system, cause the computing system to perform a method comprising:
receiving a diagnostic exam at a diagnostic exam review system from a data source;
assigning the diagnostic exam to a first medical professional for interpretation;
communicating the diagnostic exam to a computing device operated by the first medical professional;
receiving a primary interpretation of the diagnostic exam at the diagnostic exam review system from the computing device operated by the first medical professional;
recording the primary interpretation of the diagnostic exam in an electronic medical record system;
determining whether the diagnostic exam should be over-read for training or scoring on the primary interpretation;
when the diagnostic exam should be over-read, assigning the diagnostic exam to a second medical professional for interpretation;
communicating the diagnostic exam to a computing device operated by the second medical professional;
receiving a primary interpretation of the diagnostic exam at the diagnostic exam review system from the computing device operated by the second medical professional;
determining, at the diagnostic exam review system, if the primary interpretation of the first medical professional agrees with the primary interpretation of the second medical professional;
when the primary interpretations of the first medical professional and second medical professional disagree, the diagnostic exam is assigned to a third medical professional for interpretation;
communicating the diagnostic exam to a computing device operated by the third medical professional;
receiving a primary interpretation of the diagnostic exam at the diagnostic exam review system from the computing device operated by the third medical professional;
determining, at the diagnostic exam review system, if the primary interpretation of the first medical professional agrees with the primary interpretation of the third medical professional;
scoring each medical professional's primary interpretation;
recording the scores in a medical professional performance data store; and
ranking each of the medical professionals based on the scores and recording the ranking in the medical professional performance data store.
